SEQUENCE LISTING (3 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 32 aa, synthetic construct
  VARIANT 10: note = D or E
  VARIANT 24: note = D-histidine or G
FVNQHLCGSXLVEALYLVCGERGXFYTPKTGY

(19) United States
(12) Patent Application Publication
(10) Pub. No.: US 2026/0174874 A1
(43) Pub. Date: Jun. 25, 2026

(54) INSULIN DERIVATIVES

(71) Applicant: USTAV ORGANICKE CHEMIE A BIOCHEMIE AKADEMIE VED CR, V. V. I., Praha 6 (CZ)

(72) Inventors: Jiri JIRACEK, Svemyslice (CZ); Irena SELICHAROVA, Velka Dobra (CZ); Marta LUBOS, Gdansk (PL); Terezie PANIKOVA, Plzen (CZ)

(21) Appl. No.: 19/126,415

(22) PCT Filed: Oct. 29, 2023
(86) PCT No.: PCT/CZ2023/050072

§ 371 Date: May 1, 2025

(30) Foreign Application Priority Data
Nov. 3, 2022 (CZ) PV 2022-455

(51) Int. Cl. A61K 47/54; A61K 9/00; A61K 31/357; A61K 38/00; A61K 47/60; C07K 14/47

(52) U.S. Cl. CPC A61K 47/542; A61K 9/0019; A61K 31/357; A61K 38/00; A61K 47/60; C07K 14/47

(57) ABSTRACT

Novel derivatives of human insulin are described that have binding affinities for the IGF-1 receptor surprisingly comparable to human IGF-1, while having higher affinities for both isoforms of the insulin receptor than human insulin. The novel insulin derivatives have D-histidine or glycine at position B24, the B chain has glycine at the C-terminus at position B31 and tyrosine at position B32, and glutamic or aspartic acid at position B10. The derivatives of the invention can be used particularly for growth and proliferation stimulation in cell cultures or for treatment of neurological disorders as of Alzheimer's disease, Huntington's and Parkinson's diseases, a cognitive loss or symptoms of syndromic autism, since one derivative could mediate both insulin and IGF-1 function.

### FIELD OF ART

The present invention relates to derivatives of human insulin in which four relatively small specific structural changes produce an unexpected synergistic effect of extremely increased binding affinity for the IGF-1 receptor, comparable to that of native IGF-1, which is accompanied by very high binding affinity for both isoforms of the insulin receptor.

### BACKGROUND ART

Insulin is a polypeptide hormone that regulates the overall metabolic homeostasis of the body. In response to higher blood glucose levels, insulin is released from β pancreatic cells into the circulation and binds to the transmembrane insulin receptor (IR), which exists in two isoforms, IR-A and IR-B. In response to insulin binding, the insulin receptor autophosphorylates and triggers a signaling cascade by phosphorylating intracellular proteins. These lead to the inhibition of glucose synthesis in the liver and to the translocation of the specific transporter GLUT-4 to the cell membrane of fat and muscle cells and to the entry of glucose from the blood into the cells. Insulin is an indispensable drug for millions of diabetic patients worldwide.

In addition to metabolic effects, insulin also acts as a growth hormone, i.e. by activating specific intracellular signaling pathways, it can stimulate the transcription of specific genes and thereby stimulate cell differentiation and proliferation (Nagao H., Proc. Natl. Acad. Sci. U.S.A. 118, 17:e2019474118).

The insulin receptor (IR) is a transmembrane glycoprotein belonging to the tyrosine kinase family. Binding of insulin causes a structural transformation of the receptor that results in autophosphorylation of intracellular tyrosine kinase subunits and phosphate transfer to intracellular proteins (Lawrence M. C., Mol. Metab. 52, 101255, 2021). IR exists in two isoforms, IR-A and IR-B, which differ only in the 12 amino acids present in IR-B near the insulin binding site but not in IR-A (Belfiore A., Endocr. Rev. 38, 379, 2017). Insulin binds to both IR isoforms with similar subnanomolar affinity.

Insulin consists of two peptide chains (A with 21 amino acids and B with 30 amino acids) connected by two disulfide bridges, and a third disulfide bridge, which is in the A chain, stabilizes the 3-D structure of the hormone:

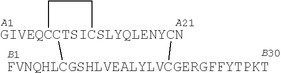

The primary sequence of human insulin, which consists of an A chain (A1-A21, SEQ ID NO. 1), a B chain (B1-B30, SEQ ID NO. 2) and three disulfide bridges (A6-A11, A7-B7 and A20-B19).

The mode of insulin binding to the insulin receptor has only been elucidated in detail in the last few years, and cryogenic electron microscopy (cryo-EM) studies have described that coordinated cooperation between two binding sites 1 and 2 in the receptor is required for insulin binding to the IR, to which insulin binds via its respective binding sites 1 and 2 (Nielsen J., J. Mol. Biol. 434, 167458, 2022). Insulin is also able to bind to the receptor for IGF-1 (IGF-1R) with about 1000-fold lower affinity than to IR-A and IR-B. The most potent ligand for IGF-1R is IGF-1 (insulin-like growth factor 1), which binds to IGF-1R with subnanomolar affinity, but to IR-A with about 100-fold and to IR-B with about 500-fold lower affinity than insulin (Jiracek J., Front. Endocrinol. 8, 167, 2017) (Table A). The binding mode of IGF-1 to IGF-1R is similar to the binding of insulin to binding site 1 of the IR (Xu Y., Nat. Commun. 9, 821, 2018), but it is assumed that IGF-1 does not use binding site 2 for binding to IGF-1R (Jiráček et al., Vitamins & Hormones 123, 187, 2023). IGF-1 and is a growth factor with an important role in the development of the organism, as well as in regenerative and healing processes and in the control of the overall growth of the organism.

Today, it is evident that brain is an insulin and IGF-1 sensitive organ where the hormones elicit multifaceted functions and regulate behavioral and metabolic responses through participation of both peripheral and local insulin hormones (Kullman S. K., Lancet Diabetes Endocrinol. 8, 524, 2020). IR and IGF-1R are expressed in different regions of the adult brain but the brain production of insulin and IGF-1 is reduced in adulthood (Fernandez A. M., Nat. Rev. Neurosci. 13, 225, 2012). The presence of IGF-1R and IR in brain vessels allows the transcytosis of the hormones to the brain through blood-brain barrier (Kadry H., Fluids Barriers CNS 17, 69, 2020). Dysfunction of insulin hormones in the brain has been described in Alzheimer's disease AD also in Huntington's and Parkinson's diseases (Benedict C., Front. Neurosci. 12, 215, 2018). Discovery that insulin can be applied to the brain through the nose (Benedict C., Psychoeneuroendocrinol. 29, 1326, 2004) has opened a way for clinical trials using intranasal insulin in aging patients as a therapeutic alternative for a cognitive loss (Craft S., JAMA Neurol. 77, 1099, 2020). IGF-1 is being evaluated as a novel treatment for core symptoms of syndromic autism in one of the first clinical trials of this kind (NCT01970345).

Published insulin modifications relevant to this patent application are summarized in Table A. We have previously found (Zakova L., J. Biol. Chem. 288, 15, 10230, 2013 and Chrudinova M., J. Biol. Chem. 293, 43, 16818) that the structural effect of D-amino acids in the B24 position of insulin is the inability of the D-amino acid side chain (due to reversed chirality) to occupy the position normally occupied by the L-Phe side chain in the B24 position of insulin. As a result, the amino acids of the B22-B30 chain are loosened and diverted away from the central hydrophobic part of insulin and PheB25 side chain occupies vacant binding site for PheB24 in insulin (so-called “downshift” effect). The loosening of the B22-B30 amino acids of insulin will facilitate their adaptation to the insulin binding site in the insulin receptor (Menting J. G., Proc. Natl. Acad. Sci. U.S.A. 111, E3395, 2014), leading to higher binding affinity of the analogues.

TABLE A
Relative binding affinities of human insulin, its published
derivatives and human IGF-1 to IR-A, IR-B and IGF-1R.

| | Relative binding | Relative binding | Relative binding |
|---|---|---|---|
| | affinity for | affinity for | affinity for |
| | IR-A (%) | IR-B (%) | IGF-1R (%) |
| Human insulin | 100 | 100 | 0.1 |
| Human IGF-1 | 1.5 | 0.2 | 100 |
| D-AlaB24-insulin | 150 | — | — |
| D-PheB24-insulin | 180 | — | — |
| D-HisB24-insulin | 280 | 250 | 1.0 |
| D-ProB24-insulin | 1.3 | — | — |
| GlyB31, TyrB32-insulin | 162 | 310 | 0.27 |
| D-HisB24, GlyB31, TyrB32-insulin | 251 | 338 | 12.4 |
| AspB10-insulin | 206 | — | 0.2* |
| GluB10-insulin | 407 | — | — |

Relative binding affinity is defined as (K_{d} of native hormone/K_{d} of derivative) × 100.
*Converted from Schaffer data (Schaffer L., Eur. J. Biochem. 221, 1127, 1994).
— indicates data not available.

Slightly increased affinity for all receptors compared to human insulin was also achieved by extending only the C-terminus of the insulin B-chain by the amino acids GlyB31 and TyrB32 in analogy to the amino acids that are in these positions in IGF-1 (Chrudinova M., J. Biol. Chem. 293, 43, 16818). Mutation of the HisB10 position for Asp or Glu in insulin itself also provided a moderate increase in receptor affinities (Schaffer L., Eur. J. Biochem. 221, 1127, 1994 and Kaarsholm N. C., Biochemistry 32, 10773, 1993).

### DISCLOSURE OF THE INVENTION

In the present invention, we demonstrate that by three modifications at the C-terminus of the B-chain of insulin and one modification at the N-terminal portion of the B-chain, a synergistic effect of the mutations and thus an extremely high affinity for IGF-1R, comparable to that of native IGF-1, can be surprisingly achieved while maintaining high binding affinities for both isoforms of IR.

The present invention shows that a human insulin derivative with D-histidine at position B24, with extension of the C-terminus of the B-chain by the amino acid Gly at position B31 and by the amino acid Tyr at position B32, and with Glu at position B10, has an unexpectedly extremely increased affinity for IGF-1R and also highly increased affinities for IR-A and IR-B. Thus, this insulin derivative can effectively exert its biological effects through binding to both the IR-A and IR-B receptors as well as the IGF-1R receptor. The derivatives having aspartic acid in the B10 position or Gly in the B24 position also showed a weaker additive effect on the affinity for IGF-1R.

One aspect of the invention are derivatives of human insulin having the formula I

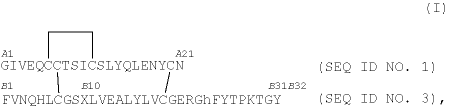

wherein the h at position B24 is D-histidine or glycine, the B chain is extended at the C-terminus by glycine at position B31 and tyrosine at position B32, and the X at position B10 is glutamic or aspartic acid.

Preferably, the invention provides the human insulin derivative according to the formula I, wherein the h at the B24 position is D-histidine, the B chain is extended at the C-terminus by glycine at the B31 position and tyrosine at the B32 position, and the X at the B10 position is glutamic acid.

Another aspect of the invention is the human insulin derivative according to the formula I, wherein the h at the B24 position is D-histidine, the B chain is extended at the C-terminus by glycine at the B31 position and tyrosine at the B32 position, and the X at the B10 position is aspartic acid.

Another aspect of the invention is also the human insulin derivative according to the formula I, wherein the h at the B24 position is glycine, the B chain is extended at the C-terminus by glycine at the B31 position and tyrosine at the B32 position, and the X at the B10 position is glutamic acid.

Another aspect of the invention is the human insulin derivative according to the formula I for use in the treatment of Alzheimer's disease, Huntington's and Parkinson's diseases, a cognitive loss, or symptoms of syndromic autism.

Yet another aspect of the invention is use of the human insulin derivatives of formula I in stimulation of cell proliferation and differentiation in cell cultures (i.e., in vitro).

Also, an object of the invention is use of the human insulin derivatives of formula I in in vitro stimulating tissue formation from germ cells.

An object of the invention is also use of the human insulin derivatives of formula I in stimulation of regeneration of cell cultures and tissues in vitro following damage thereto.

The herein described derivatives of human insulin have binding affinities for the IGF-1 receptor surprisingly comparable to human IGF-1, while having higher affinities for both isoforms of the insulin receptor than human insulin. They could be advantageously used particularly for growth and proliferation stimulation in cell cultures or for treatment of neurological disorders as of Alzheimer's disease, Huntington's and Parkinson's diseases, a cognitive loss, or symptoms of syndromic autism, preferably after intranasal application.

### BRIEF DESCRIPTION OF DRAWINGS

FIG. 1A,B,C,D shows the binding curves for IR-A. These represent the inhibition of the binding of human [¹²⁵I]-monoiodotyrosyl-insulin to IR-A by human insulin (HI, FIG. 1A), the derivative IN-ML-9 (FIG. 1B), the derivative IN-ML-55 (FIG. 1C) and the derivative IN-ML-15 (FIG. 1D).

FIG. 2A,B,C,D shows the binding curves for IR-B. These are the inhibition of the binding of human [¹²⁵I]-monoiodotyrosyl-insulin to IR-B by human insulin (HI, FIG. 2A), the derivative IN-ML-9 (FIG. 2B), the derivative IN-ML-55 (FIG. 2C) and the derivative IN-ML-15 (FIG. 1D).

FIG. 3A,B,C,D,E shows the binding curves for IGF-1R. These are the inhibition of the binding of human [¹²⁵I]-monoiodotyrosyl-IGF-1 to IGF-1R by human IGF-1 (FIG. 3A), the derivative IN-ML-9 (FIG. 3B), the derivative IN-ML-55 (FIG. 1C) the derivative IN-ML-15 (FIG. 3D) and human insulin (FIG. 3E).

FIG. 4 shows the concentration dependence of the rate of autophosphorylation of IGF-1R, IR-A and IR-B receptors by human insulin (HI), insulin-like growth factors (IGF-1 and IGF-2) and IN-ML-9 and IN-ML-15 derivatives. Data are expressed relative to the signal elicited by treatment with 10 nmol·l⁻¹ insulin (IR-A and IR-B) or 10 nmol·l⁻¹ IGF-1 (IGF-1R).

FIG. 5A,B shows the effect of insulin (HI), insulin-like growth factors (IGF-1 and IGF-2), and the IN-ML-15 derivative at 10 nmol·l⁻¹ (right) and 1 nmol·l⁻¹ (left) concentrations on SH-SY5Y cell growth (FIG. 5A). FIG. 5B shows the effect of insulin (HI), insulin-like growth factors (IGF-1 and IGF-2) and the IN-ML-15 derivative at 10 nmol·l⁻¹ (right) and 1 nmol·l⁻¹ (left) concentrations on suppressing the toxic effect of methylglyoxal on SH-SY5Y cell growth. Data are relative to the number of cells in basal medium without added serum (basal). Statistically significant change in cell growth compared to cells in the presence of 1.5 mmol·l⁻¹ methylglyoxal (MG) calculated by ANOVA (“analysis of variance”) is expressed as * p<0.05, ** p<0.01, *** p<0.001.

### EXAMPLES

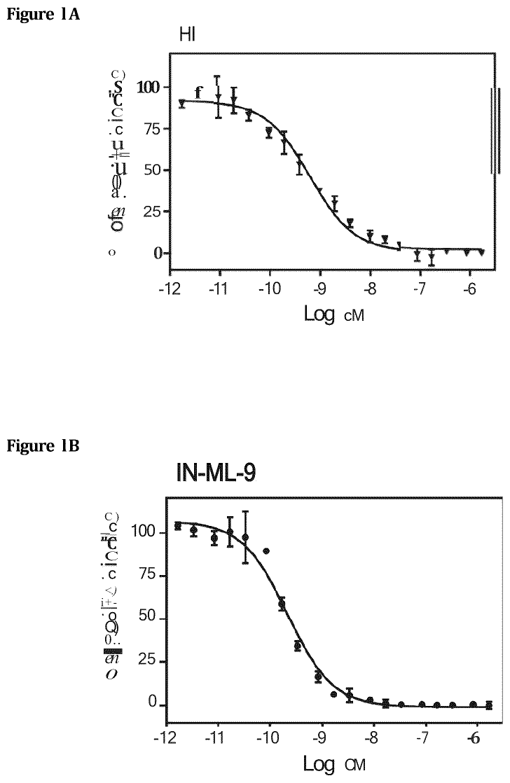

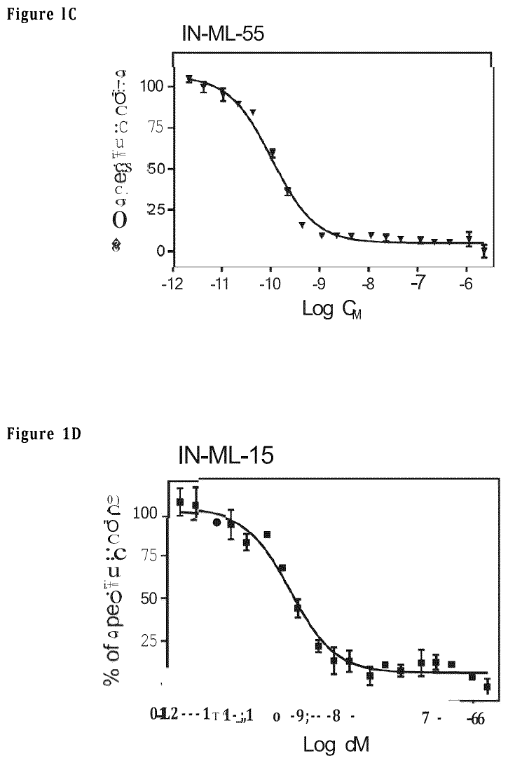

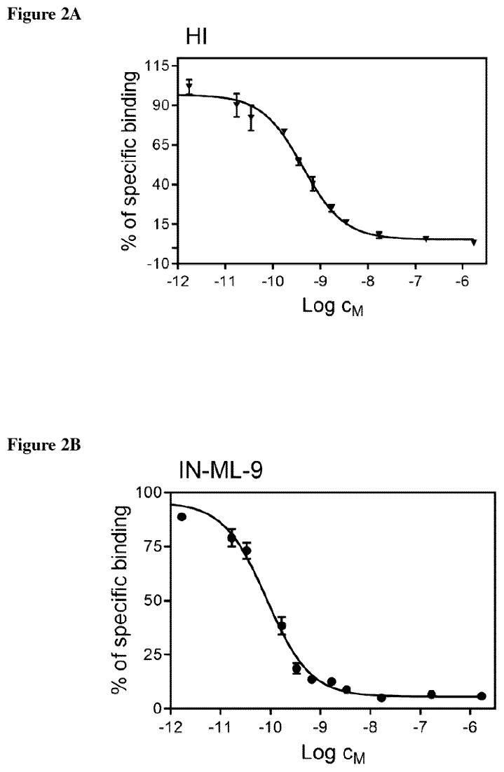

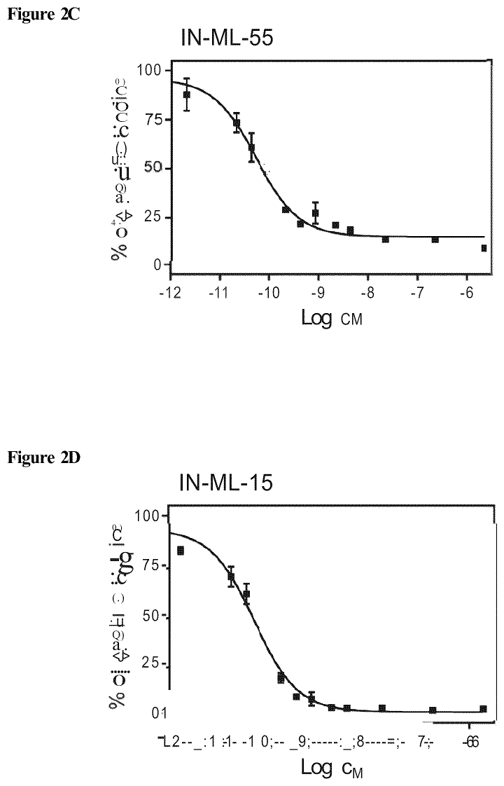

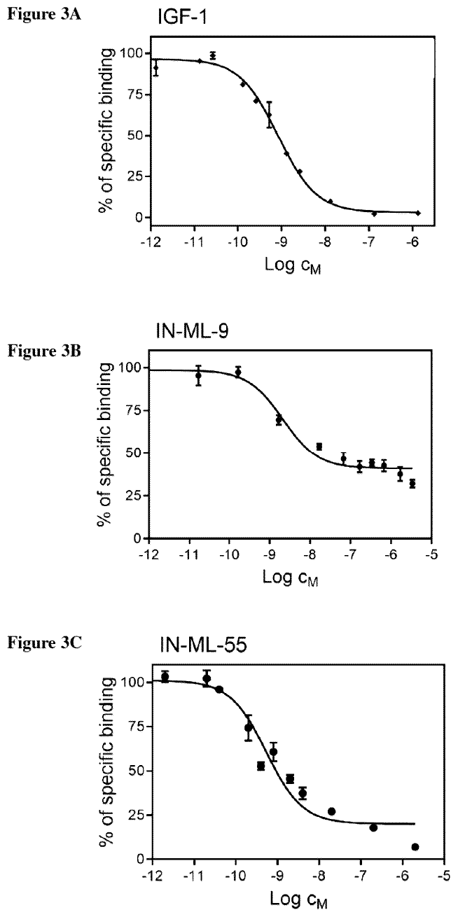

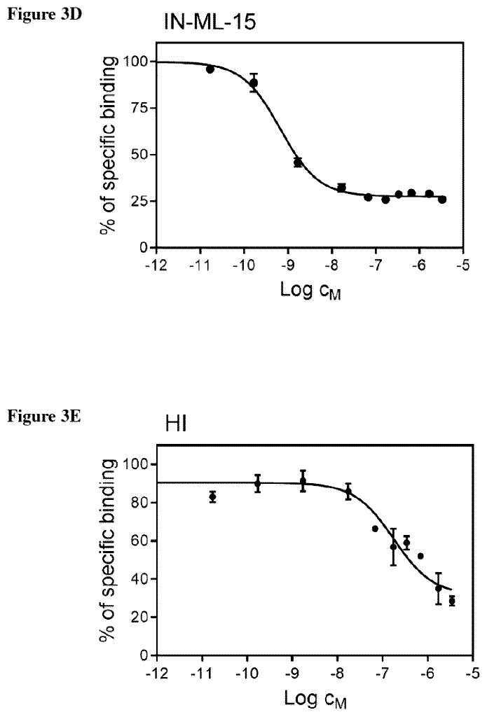

Three new insulin analogues were prepared in which the following mutations were combined: D-HisB24, GlyB31, TyrB32 and either AspB10 in analogue IN-ML-9 or GluB10 in analogue IN-ML-15, and analogue IN-ML-55 has GlyB24, GluB10 and GlyB31 and TyrB32. The binding affinities of the analogues are shown in Tables 1-3 and the binding curves in FIGS. 1-3. Surprisingly, it turns out that the effects of the individual substitutions in insulin add up, are synergistic, and the result is that all three derivatives are multiple times more potent in binding to IR-A and IR-B than human insulin and at the same time, and most importantly, have very high affinities for IGF-1R, making them the most potent known insulin analogues for this receptor. The binding affinity of IN-ML-9 for IGF-1R is 18% of human IGF-1 and the binding affinity of IN-ML-55 is 30% of human IGF-1 (the binding affinity of human insulin is only 0.1% of IGF-1). However, the extremely high binding affinity of IN-ML-15 (with GluB10) to IGF-1R (79% of human IGF-1) is unprecedented and already quite comparable to the affinity of native IGF-1 to IGF-1R. The dramatic increase from 18% to 79% due to the extension of the amino acid side chain in B10 by a single CH₂ group (Asp to Glu) was completely unpredictable.

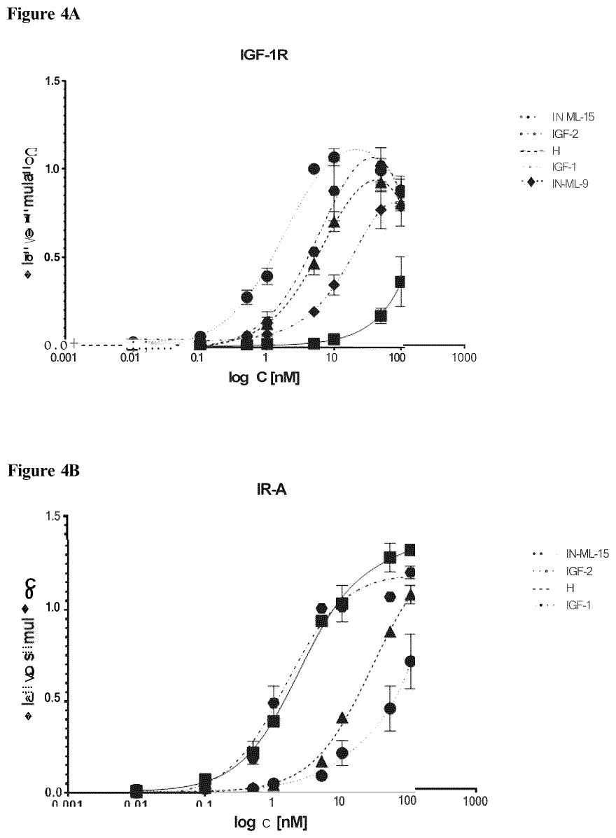

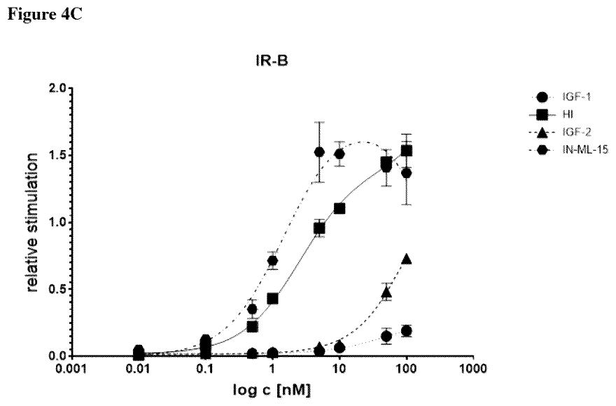

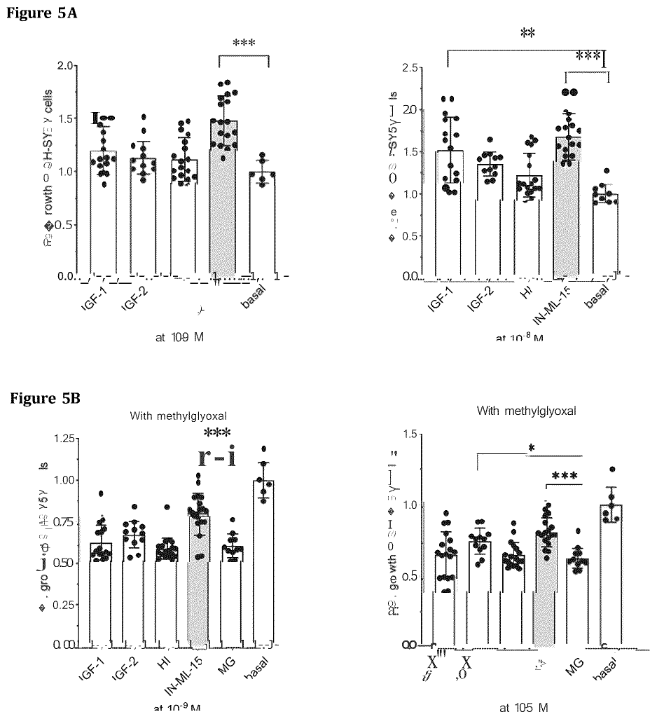

The high affinity of IN-ML-9 and especially IN-ML-15 derivatives for receptors was reflected in their ability to activate IR-A, IR-B and IGF-1R receptors (FIG. 4). The high binding affinity of the IN-ML-15 derivative was also reflected in its ability to stimulate the growth of SH-SY5Y cells (FIG. 5A) and even in its ability to attenuate the toxic effect of methylglyoxal on these cells (FIG. 5B). This effect is explained precisely by the potent action of the analogue on receptors for both insulin and IGF-1, mediating the action of both insulin and IGF-1 simultaneously.

A unique feature of IN-ML-9 and especially IN-ML-15 derivatives is their extremely high affinity for the IGF-1 receptor (18% and even 79% of native IGF-1, which contrasts with the 0.1% binding affinity of human insulin). This characteristic, while maintaining a high affinity for the receptor for insulin, makes IN-ML-15 an ideal candidate for use where the growth potential of both insulin and IGF-1 is needed, which may be, for example, media for stimulating cell growth and differentiation, and where a single derivative (IN-ML-15) could replace the action of two native hormones with greater efficiency and at lower cost.

List of abbreviations

| | |
|---|---|
| DIC | N,N′-dicyclohexylcarbodiimide |
| Fmoc | fluorene-9-ylmethoxycarbonyl |
| HBTU | O-(benzotriazol-1-yl)-N,N,N′,N′- |
| | tetramethyluroniumhexafluorine phosphate |
| Tris | tris(hydroxymethyl)aminomethane |
| S.D. | Standard deviation |
| ANOVA | Analysis of variation, analysis of variance |
| BSA | bovine serum albumin |
| HOBt | hydroxybezotriazole |
| DIPEA | N,N-diisopropylethylamine |
| DMF | dimethylformamide |
| RT | Retention time |
| tBu | tertiary butyl |
| DCM | dichloromethane |
| TFA | trifluoroacetic acid |
| TIS | triisopropylsilane |
| Et₂O | diethyl ether |
| HPLC | High performance liquid chromatography |
| v/v | Volume ratio |
| AcOH | acetic acid |
| MTT assay | Colorimetric assay for measuring cellular |
| | metabolic and proliferative activity |
| MS | Mass spectrometry |
| HR | High-resolution |
| ESI | Electrospray ionization |
| MALDI | Matrix-assisted laser desorption/ionization |

### Example 1: Synthesis of Insulin Derivatives

We used a strategy of total chemical synthesis of insulin using orthogonal protection of the SH groups of individual cysteines. For the preparation of IM-ML-9, IN-ML-55 and IN-ML-15 derivatives, we used the procedure according to Liu et al. (Liu F. Angew. Chem. Int. Ed., 3983, 2014).

The insulin chains were synthesized by solid-phase synthesis on a Spyder Mark IV Multiple Peptide Synthesizer (European Patent application EP17206537.7) developed at the Development Center of the IOCB using Rink Amide AM resin for chain A and Fmoc-Tyr(tBu)-Wang resin for chain B.

### Chain A

The first A-chain amino acid, Fmoc-Asp-OtBu, was manually attached to the Rink Amide AM resin using the B-carboxy group of the side chain. The amino acids in positions A6 to A11 (Fmoc-Cys(StBu)-OH for position A6, Fmoc-Cys(Acm)-OH for position A7, the isoacyl dipeptide Boc-Ser [Fmoc-Thr(tBu)]-OH for positions A8 and A9, Fmoc-Ile-OH for position A10 and Fmoc-Cys(Mmt)-OH for position A11) were also manually attached to the resin. The use of isoacyl dipeptide significantly increases the solubility of the protected peptide chain. For the condensation reaction, 3 equivalents of the protected amino acid were used, 3 equivalents of the protected amino acid were used. HBTU, 3 eq. HOBt and 6 eq. DIPEA in DMF for 2 h and the reactions were controlled by the Kaiser test. The protecting group Fmoc was cleaved using 20% piperidine in DMF (2 and 20 min). The remaining amino acids were condensed onto resin using an automated synthesizer under identical reaction conditions but without control by the Kaiser test.

After peptide synthesis, the resin was reacted with 25% B-mercaptoethanol in DMF for 1.5 h at RT. After 1.5 h, the reaction was repeated. By analyzing a small sample of the resin, it was checked whether the protecting tBu group on Cys at the A6 position was cleaved. The resin was then washed with DMF and DCM and treated with 10 eq DTNP (2,2′-Dithiobis(5-nitropyridine) in DCM for 1 h at RT. After reaction, the resin was washed with DMF and DCM and reacted with 1% TFA, 5% TIS in DCM for 5×2 min at RT. Then the resin was washed with DMF and DCM and stirred in DCM for 1 h at RT. The resin was then treated with TFA/TFA/H2O (95/2.5/2.5) for 1.5 h, the peptide was precipitated from the solution with cold Et2O, and purified by HPLC on a Nucleosil 100-7 C8 column (250×10 mm, 7 μm, Macherey-Nagel) on a Waters HPLC system (Waters 600 with 2487 Dual λ Absorbance Detector) at a flow rate of 4 ml/min in a gradient of acetonitrile in water with 0.1% TFA (solvent A: 0.1% TFA (v/v) in H2O; solvent B: 0.1% TFA (v/v) in 80% CH3CN, t=0 min at 10% B, t=30 min at 100% B). The compounds were detected at 218 nm. The purity of the compounds was checked on a Nucleosil 120-5 C8 column (250×4.6 mm, 5 μm, Macherey-Nagel) at a flow rate of 1 ml/min using the same gradient and solutions on a Watrex HPLC system (Watrex DeltaChrom™ P200 binary Pump and Wufeng LC-100 UV Detector. The identity of the compounds was confirmed by mass spectrometry.

### Chain B

Chain B was prepared in an automated peptide synthesizer using the same reagents as chain A and was also cleaved, purified and analyzed as above.

### Ligation of A and B

The A and B chains of the analogues were mixed in 6 mol·l⁻¹ urea and 0.2 mol·l-1 NH₄HCO₃ buffer (pH 8). The mixture was stirred until completely dissolved and after 5 min in RT, 25 eq. (relative to chain A) of freshly prepared iodine solution in AcOH. The resulting solution was stirred gently for 10 min in RT and then a 1 mol·l⁻¹ solution of ascorbic acid was added until the solution was decoloured. The solution was then evaporated, and the derivatives purified using RP-HPLC as described above.

The purity (>95%) of final derivatives were checked by RP-HPLC monitored at 218 nm. The identity of derivatives was confirmed by HR MS.

### Example 2: Binding Affinity of Derivatives for IR-A, IR-B and IGF-1R Receptors

The binding affinities of the derivatives for the insulin receptor isoform A (IR-A) were determined by competition of the derivatives with radiolabeled 125I-insulin for the insulin receptor IR-A in IM-9 lymphocytes according to Morcavallo et al. (Morcavallo A., J. Biol. Chem., 11422, 2012). Binding affinity for IR-B was determined using mouse fibroblasts transfected with human IR-B and with deleted mouse IGF-1R according to Zakova et al. (Zakova L., Acta Crystallogr. D, 2765, 2014). Binding affinities to IGF-1R were determined by co-precipitation of radiolabeled 125I-IGF-1 derivatives with human IGF-1R in mouse fibroblasts transfected with human IGF-1R and with deleting mouse IGF-1R according to Hexnerová et al. (Hexnerová R., J. Biol. Chem. 291, 21234, 2016).

The binding affinities of the selected derivatives are shown in Tables 1 to 3 and representative binding curves are shown in FIGS. 1 to 3. Individual binding curves for each derivative or human insulin were constructed from duplicate points and the final dissociation constant (K_{d}) was calculated from at least three (n=3-5) independent binding curves (each curve providing one K_{d} value) independently. By combining four mutations in insulin, each of which was known to increase binding affinity for IR-A, IR-B and IGF-1R, an additive effect was achieved, resulting in the novel derivatives IM-ML-9, IN-ML-55 and IN-ML-15, which have about twice to four-fold (for IN-ML-55) the binding affinity of human insulin for IR-A (Table 1). The binding affinities of both new derivatives towards IR-B are even higher; IN-ML-9 and IN-ML-55 achieve almost fivefold (463% and 48%, respectively) binding affinity relative to human insulin and IN-ML-15 even almost tenfold (950%) affinity of human insulin (Table 2). The binding affinities of the new derivatives toward IGF-1R are extremely high, as IN-ML-9 and IN-ML-55 achieve nearly 18% and 30% IGF-1 affinity, respectively, which is higher than the most potent insulin derivative to date, our published D-HisB24, GlyB31, TyrB32-insulin (12% IGF-1). However, the affinity of IN-ML-15 is unprecedented as it achieves 79% of the binding of native IGF-1 to IGF-1R (790 times more potent than insulin), making it almost equipotent with IGF-1.

TABLE 1
Binding affinity of human insulin and its derivatives for IR-A in human IM-9 lymphocytes.

| | K_{d} ± S.D. nmol · l⁻¹ | Relative binding |
|---|---|---|
| | (n = 3)¹ | affinity (%)² |
| Human insulin | 0.27 ± 0.08^{a} | 100 |
| | 0.46 ± 0.09^{b} | 100 |
| | 0.40 ± 0.01^{c} | 100 |
| IN-ML-9 (AspB10, D-HisB24, GlyB31, TyrB32-insulin) | 0.14 ± 0.01^{a} | 193 |
| IN-ML-55 (GluB10, GlyB24, GlyB31, TyrB32-insulin) | 0.10 ± 0.01^{c} | 400 |
| IN-ML-15 (GluB10, D-HisB24, GlyB31, TyrB32-insulin) | 0.22 ± 0.07^{b} | 209 |

¹The K_{d} of human insulin and its derivatives for IR-A was determined in three independent series of measurements (indexed ^{a, b} or ^{c}). The individual K_{d} values of the ligands in this column are relative to the corresponding native insulin K_{d} value (e.g., ^{a} to ^{a}, etc.). n is the number of repetitions.
²Relative binding affinity is defined as (K_{d} of native insulin/K_{d} of derivative) × 100 (%).

TABLE 2
Binding affinity of human insulin and its derivatives for IR-B in mouse fibroblasts.

| | K_{d} ± S.D. nmol · l⁻¹ | Relative binding |
|---|---|---|
| | (n = 3)¹ | affinity (%)² |
| Human insulin | 0.37 ± 0.02^{a} | 100 |
| | 0.38 ± 0.14^{b} | 100 |
| | 0.24 ± 0.03^{c} | 100 |
| IN-ML-9 (AspB10, D-HisB24, GlyB31, TyrB32-insulin) | 0.08 ± 0.02^{a} | 463 |
| IN-ML-55 (GluB10, GlyB24, GlyB31, TyrB32-insulin) | 0.05 ± 0.01^{c} | 480 |
| IN-ML-15 (GluB10, D-HisB24, GlyB31, TyrB32-insulin) | 0.04 ± 0.01^{b} | 950 |

¹The K_{d} of human insulin and its derivatives for IR-B was determined in three independent series of measurements (indexed ^{a, b} or ^{b}). The individual K_{d} values of the ligands in this column are relative to the corresponding native insulin K_{d} value (e.g., ^{a} to ^{a}, etc.). n is the number of repetitions.
²Relative binding affinity is defined as (K_{d} of native insulin/K_{d} of derivative) × 100 (%).

TABLE 3
Binding affinity of human IGF-1, human insulin and
its derivatives to IGF-1R in mouse fibroblasts.

| | K_{d} ± S.D. nmol · l⁻¹ | Relative binding |
|---|---|---|
| | (n = 3)¹ | affinity (%)² |
| Human IGF-1 | 0.30 ± 0.06^{a} | 100 |
| | 0.13 ± 0.03^{b} | |
| Human insulin | 292 ± 31^{a} | 0.1 |
| IN-ML-9 (AspB10, D-HisB24, GlyB31, TyrB32-insulin) | 1.67 ± 0.31^{a} | 18 |
| IN-ML-55 (GluB10, GlyB24, GlyB31, TyrB32-insulin) | 0.43 ± 0.04^{b} | 30 |
| IN-ML-15 (GluB10, D-HisB24, GlyB31, TyrB32-insulin) | 0.38 ± 0.14^{a} | 79 |

¹The K_{d} of human IGF-1, its derivatives and human IGF-1 for IGF-1R was determined in two independent series of measurements (indexed ^{a} or ^{b}). The individual K_{d} values of the ligands in this column are relative to the corresponding native insulin K_{d} value (e.g., ^{a} to ^{a}, etc.). n is the number of repetitions.
²Relative binding affinity is defined as (K_{d} of native insulin/K_{d} of derivative) × 100 (%).

### Example 3. Autophosphorylation of IR-A, IR-B and IGF-1R Receptors after Stimulation with Derivatives

Autophosphorylation of insulin and IGF-1 receptors was detected using a specific antibody against phosphorylated tyrosines in the receptor kinase domain (anti-phospho-IGF-1Rβ (Tyr1135/1136)/IRβ (Tyr1150/1151), Cell Signaling) using the methodology described in (Macháčková 2017). Mouse fibroblasts transfected with human IR-A, IR-B or human IGF-1R and with deleted mouse IGF-1R were grown in 96-well plates. After 4 h in serum-free medium, cells were stimulated with natural ligands or derivatives at increasing concentrations for 20 min. Cells were fixed with formaldehyde and cell membranes were disrupted with 0.1% Triton-X-100. Possible non-specific sites of protein sorption in the wells were blocked with 5% BSA (bovine serum albumin). After incubation with the antibody, the level of receptor autophosphorylation was detected by measuring horseradish peroxidase-catalyzed luminol chemiluminescence, which was labeled with a secondary antibody. The concentration dependence of autophosphorylation of IR-A, IR-B, and IGF-1R is shown in FIG. 4. It is clear from the Figure that the increased binding of the derivatives to the individual receptors is reflected in their stimulation, i.e. the binding effect is reflected in the biological activity of the derivatives.

### Example 4. Effect of IN-ML-15 Derivative on the Growth of SY5Y Cell Line without or in the Presence of Methylglyoxal

The ability of the IN-ML-15 derivative to stimulate cell proliferation (growth) was studied in the neuroblastoma cell line SY5Y (Zmeškalová A. Int. J. Mol. Sci 21, 6343, 2020). Cells were grown in 96-well plates. After 24 hours in serum-free growth medium, natural ligands or the IN-ML-15 derivative were added at 1 nmol·l-1 or 10 nmol·l-1 concentration, and after a further 20 hours, cell growth over control was determined by MTT assay. The results are shown in FIG. 5A. The IN-ML-15 derivative significantly stimulates cell growth. The ability of the derivative to prevent cell death caused by methylglyoxal was measured in a similar manner, except that after the addition of the derivative, methylglyoxal was added to the cells at a final concentration of 1.5 mmol·l-1. Methylglyoxal at this concentration caused a reduction in the amount of cells grown in the wells to approximately 50-60% compared with the control. From the results shown in FIG. 5B, it is clear that the IN-ML-15 derivative was able to prevent cell death.

### INDUSTRIAL APPLICABILITY

The human insulin derivative according to the present application of the invention has higher binding affinities towards both isoforms of IR, and at the same time has an extremely high binding affinity towards IGF-1R. Therefore, it can be advantageously used especially in cell culture for stimulating cell growth and differentiation. The human insulin derivative could be also used for treatment for symptoms of neural disorders manifested by a cognitive loss where derivative can be applied to the brain through the nose.

1. An insulin derivative of formula I 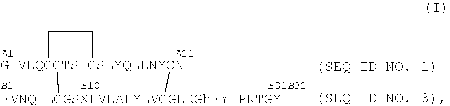
wherein
h at position B24 is D-histidine or glycine,
the B chain has glycine at the C-terminus at position B31 and tyrosine at position B32, and
X at position B10 is glutamic acid or aspartic acid.

2. The insulin derivative of formula I according to claim 1, wherein the h at position B24 is D-histidine and the X at position B10 is glutamic acid.

3. The insulin derivative of formula I according to claim 1, wherein the h at position B24 is D-histidine and the X at position B10 is aspartic acid.

4. The insulin derivative of formula I according to claim 1, wherein the h at position B24 is glycine and the X at position B10 is glutamic acid.

5. A method of administering a medicament comprising the insulin derivative of formula I according to claim 1 for treatment to a subject in need thereof.

6. A method of administering the insulin derivative of formula I according to claim 1 for treatment of Alzheimer's disease, Huntington's and Parkinson's diseases, cognitive loss, or symptoms of syndromic autism.

7. A method of administering the insulin derivative of formula I according to claim 1 in in vitro stimulating cell growth and differentiation.

8. A method of administering the insulin derivative of formula I according to claim 1 in in vitro stimulating tissue formation from germ cells.

9. A method of administering the insulin derivative of formula I to claim 1 in in vitro stimulating regeneration of cell cultures and tissues following damage thereto.
